(19) United States
(12) Patent Application Publication
(10) Pub. No.: US 2023/0271930 A1
(43) Pub. Date: Aug. 31, 2023

(54) PROCESS FOR ISOLATION AND PURIFICATION OF THCA FROM CANNABIS

(71) Applicant: Protonify Corporation, Ottawa (CA)

(72) Inventors: David A. FISHER, Ottawa (CA); Jason M. ROSE, Ottawa (CA); Andrew FISHER, Ottawa (CA)

(21) Appl. No.: 18/009,029

(22) PCT Filed: Jun. 8, 2021
(86) PCT No.: PCT/CA2021/050781

§ 371 Date: Dec. 8, 2022

Related U.S. Application Data
(60) Provisional application No. 63/036,636, filed on Jun. 9, 2020.
(60) Provisional application No. 63/131,587, filed on Dec. 29, 2020.

(51) Int. Cl. C07D 311/80; B01D 11/02; B01D 11/04; B01D 15/42

(52) U.S. Cl. CPC C07D 311/80; B01D 11/0288; B01D 11/0492; B01D 11/0284; B01D 11/0488; B01D 15/426; B01D 11/048

(57) ABSTRACT

Processes are described for obtaining highly purified tetrahydrocannabinolic acid (THCa) from Cannabis. Solvent extraction is performed on plant material or extract, followed by removal of impurities using sequential liquid-liquid extractions to purify cannabinoid carboxylic acids therefrom based on chemical properties of carboxylate salts. The product liquor, comprising THCa in solvent, is largely free of impurities, and high in THCa. Further steps can be conducted to obtain a highly enriched solution using chromatography and subsequent crystallization of THCa in 99% purity. THCa can be used as starting material for other products that include THC by decarboxylation. Optionally, triglyceride extraction of a washed aqueous phase can be used to prepare a THCa composition without chromatographic purification. A pre-processing aqueous extraction with pH manipulations may be used to remove biomass prior to solvent extraction, while maintaining THCa and optionally other cannabinoid acids.

### CROSS REFERENCE TO RELATED APPLICATIONS

This application claims the benefit of and priority to U.S. Provisional Patent Application No. 63/036,636 entitled PROCESS FOR ISOLATION AND PURIFICATION OF THCA FROM CANNABIS filed Jun. 9, 2020, and U.S. Provisional Patent Application No. 63/131,587 entitled PROCESS FOR ISOLATION AND PURIFICATION OF THCA FROM CANNABIS filed Dec. 29, 2020, the contents of which are both hereby incorporated by reference.

### FIELD

The present disclosure relates generally to cannabinoid isolation and purification from plant material. More particularly, the present disclosure relates to a process for purification of tetrahydrocannabinolic acid (THCa) from Cannabis.

### BACKGROUND

Cannabinoids are a classification of compounds found in plants of the Cannibaceae family, the most recognizable of which is Cannabis sativa (C. sativa), but which also includes others such as C. indicia. The Cannibaceae family of plants is herein referenced interchangeable as the Cannabis plant. Cannabinoids may be interchangeably referred to as phytocannabinoids, when produced in plants.

Extraction and purification of different cannabinoids is desirable, in order to produce consumer products of known and dependable composition, such as may be required for regulatory purposes. Extraction and purification technologies generally permit isolation of different types of cannabinoids from Cannabis plants. Efficiencies of such processes will assist in achieving consumer products at competitive price points. While typically more expensive than extraction from natural sources, synthetically production of cannabinoids is possible, via laboratory processes. Synthetic processes may permit reliable production of desired cannabinoids possessing the structure and function of phytocannabinoids, but the expense for the required amounts renders synthetic methods impractical.

The most well-known psychoactive cannabinoid is delta-9-tetrahydrocannabinol (THC). Tetrahydrocannabinolic acid (THCa) is a plant-derived cannabinoid that resembles the structure of THC, but does not possess psychoactive effects in humans. While THCa serves as a precursor to THC, and occurs naturally within plants, the human body does not possess conversion enzymes to permit consumed THCa to convert to THC to achieve psychoactive effects. Heat catalysis may be used to remove the carboxyl group from THCa to form THC.

THCa is generally found in small quantities in C. sativa, of about 1-22% by dry weight, relative to other major cannabinoids. The quantity of THCa produced depends upon factors such as growing conditions and plant strain. Other strains and varieties, such as C. indicia, may also be utilized to obtain cannabinoids.

While human consumption of THCa does not result in psychoactive effects, (often referenced as being “high”), other desirable physiological effects have been associated with THCa, including: analgesic properties for pain management and relief, antiemetic properties, mitigation of appetite loss, anti-inflammatory effects, reduced insomnia, and enhanced sexual performance. While the non-psychoactive nature of THCa may render this cannabinoid of little interest to recreational Cannabis users, THCa may be more popular among consumers desirous of medicinal effects.

Cannabinoid receptors located throughout the body, are part of the endocannabinoid system, which is involved in a variety of physiological processes including appetite, pain-sensation, mood, and memory.

THCa is a Cannabis compound that is beginning to demonstrate immense therapeutic potential despite the infancy of its research. Our unique materials process isolates and extracts pure THCa as a safe and practical ingredient to alter your brands end user experience.

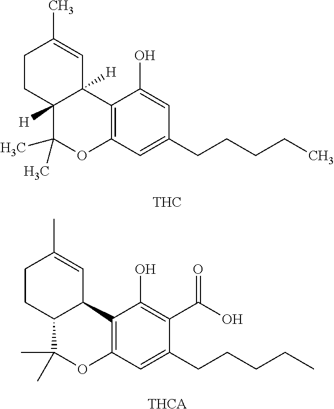

Cannabinoid receptors are of a class of cell membrane receptors in the G protein-coupled receptor superfamily. Humans have two main cannabinoid receptors by which certain effects of cannabinoids are realized, Cannabinoid Receptor type 1 (CB1) and Cannabinoid Receptor Type 2 (CB2). THC binds to CB1 as a receptor agonist, resulting in particular psychoactive effects of THC. THCa is not shown to be a strong agonist of CB1, and thus does not result in the psychoactive effect observed with THC.

Obtaining purified THCa through an extraction and/or isolation process is desirable for consumers and to further the investigations into beneficial health properties. Known methods for extraction and isolation include extractions in n-propane such as taught in Patent Publication US2018/344785A1; hexane-based extractions such as taught in Patent Publication US2019/177294A1; as well as other processes involving extraction, crystallization, and filtration, such as taught in Patent Publications US2017008870A1 and US2018/273501A1, all of which are hereby incorporated by reference. It is desirable to provide alternatives to and/or improvements upon existing processes and technologies so as to provide consumers with purified products in an economical manner.

### SUMMARY

It is an object of the present disclosure to mitigate disadvantage of previous THCa purification processes, and/or provide alternatives thereto.

A process is described herein for purification of tetrahydrocannabinolic acid (THCa) from Cannabis plant material comprising the steps of: (a) preparing a mother liquor of finely divided Cannabis plant material in a C5-C8 hydrocarbon solvent with about 0.1% acid by volume; (b) washing the mother liquor with an aqueous solution; (c) treating the washed mother liquor with a weak metal ion solution to form THCa metal salt; (d) extracting the treated mother liquor of (c) with a basic solution of NaOH at pH 12-14 to obtain THCa in aqueous phase; (e) washing the aqueous phase of (d) with the C5-C8 hydrocarbon solvent, and obtaining the aqueous phase; (f) acidifying the aqueous phase obtained in (e) to pH 3-6 to remove metal ion from THCa; and (g) extracting the acidified aqueous phase with the C5-C8 hydrocarbon solvent to obtain a solvent phase rich in THCa from which purified THCa is removed.

Further, a process is described herein for purification of tetrahydrocannabinolic acid (THCa) from Cannabis plant material comprising the steps of: milling Cannabis plant material to form a finely divided Cannabis plant material; extracting the finely divided Cannabis plant material with a C6 or C7 hydrocarbon solvent comprising 0.1% glacial acetic acid to produce a mother extraction liquor; reducing the mother extraction liquor under reduced atmosphere to remove 75% of the solvent, thereby producing a reduced mother liquor; washing the reduced mother liquor with distilled water and separating the phases to obtain a washed mother liquor; treating the washed mother liquor with an aqueous 5% sodium bicarbonate solution and separating the phases to obtain a treated mother liquor; extracting the treated mother liquor with a 0.1 M solution of NaOH and separating the phases to obtain an aqueous solution comprising THCa sodium carboxylate salt; washing the aqueous solution comprising THCa sodium carboxylate salt three times with the C6 or C7 hydrocarbon solvent and separating the phases to obtain an aqueous solution of washed THCa sodium carboxylate salt; acidify the aqueous solution of washed THCa sodium carboxylate salt to pH 2.5-4, layer with the C6 or C7 hydrocarbon solvent and separating the resulting phases to obtain an organic solution comprising THCa salt; and washing the organic solution comprising THCa salt with distilled water and separating the phases to obtain a solvent phase rich in THCa.

A process is described for preparation of a triglyceride oil comprising tetrahydrocannabinolic acid (THCa) from Cannabis plant material comprising the steps of: (a) preparing a mother liquor from Cannabis plant material extracted with a C5-C8 hydrocarbon solvent with 0.005% to 0.2% acid by volume; (b) washing the mother liquor with an aqueous solution; (c) treating the washed mother liquor with a weak metal ion solution to form THCa metal salt; (d) extracting the treated mother liquor of (c) with a basic solution of NaOH at pH 12-14 to obtain THCa in aqueous phase; (e) washing the aqueous phase of (d) with the C5-C8 hydrocarbon solvent, and obtaining the washed aqueous phase; and (f) extracting the washed aqueous phase of step (e) with a triglyceride oil to obtain the triglyceride oil comprising THCa.

A process for purification of tetrahydrocannabinolic acid (THCa) from Cannabis plant material is described, comprising the steps of: milling Cannabis plant material to form a finely divided Cannabis plant material; extracting the finely divided Cannabis plant material with a C6 or C7 hydrocarbon solvent comprising 0.1% glacial acetic acid to produce a mother extraction liquor; reducing the mother extraction liquor under reduced atmosphere to remove 75% of the solvent, thereby producing a reduced mother liquor; washing the reduced mother liquor with distilled water and separating phases to obtain a washed mother liquor; treating the washed mother liquor with an aqueous 5% sodium bicarbonate solution and separating the phases to obtain a treated mother liquor; extracting the treated mother liquor with a 0.1 M solution of NaOH and separating phases to obtain an aqueous solution comprising THCa sodium carboxylate salt; washing the aqueous solution comprising THCa sodium carboxylate salt three times with the C6 or C7 hydrocarbon solvent and separating to obtain an aqueous solution of washed THCa sodium carboxylate salt; and extracting the solution with a triglyceride oil to obtain the oil comprising THCa.

Other aspects and features of the present disclosure will become apparent to those ordinarily skilled in the art upon review of the following description of specific embodiments in conjunction with the accompanying figures.

### BRIEF DESCRIPTION OF THE DRAWINGS

Embodiments of the present disclosure will now be described, by way of example only, with reference to the attached Figures.

FIG. 1 is a flowchart representing the steps in an embodiment of the process, as described herein.

FIG. 2 shows reactions for forming carboxylic acid salts sodium carboxylate (Panel A) and ammonium carboxylate (Panel B).

FIG. 3 is a flowchart representing the optional pretreatment steps for Cannabis plant material prior to directing to preparation of a mother liquor.

FIG. 4 shows steps involved in a process for forming a triglyceride oil rich in THCa.

FIG. 5 is a flowchart representing the steps in an embodiment of the process of isolation and subsequent purification of THCa from dried Cannabis.

### DETAILED DESCRIPTION

The present disclosure provides a process for extraction and purification of acid form cannabinoids, such as THCa, from Cannabis.

A process is described for isolating, in high purity, THCa cannabinoid using a hydrocarbon solvent extraction of plant material comprising cannabinoids, terpenes, lignins, waxes and other impurities. A hydrocarbon solvent extraction is performed on dried plant material, followed by removing impurities using sequential liquid-liquid extractions to purify cannabinoid carboxylic acids from the mixture based on the chemical properties of carboxylate salts. The product liquor, comprising THCa in hydrocarbon solvent, is largely free of neutral and acidic or basic impurities, and adequately concentrated to be purified further, such as by chromatography. It is then possible to separate chemically similar acids or bases using the chromatography profile. Following the liquid-liquid extraction, work-up and washing steps render a higher quality solution, containing higher levels of purity of THCa cannabinoid than the starting solvent extract. Processes are also described for obtaining an even higher enriched solution of THCa from chromatography, and subsequent crystallization of THCa with a high level of purity, such as 99% or greater. Crystallized THCa can be used as a starting material for other reactions or products that include THC by means of decarboxylation, as described herein.

A process for purification of tetrahydrocannabinolic acid (THCa) from Cannabis plant material is described herein. The process comprises the steps of: (a) preparing a mother liquor of finely divided Cannabis plant material in a C5-C8 hydrocarbon solvent with about 0.1% acid by volume; (b) washing the mother liquor with an aqueous solution; (c) treating the washed mother liquor with a weak metal ion solution to form THCa metal salt; (d) extracting the treated mother liquor of (c) with a basic solution of NaOH at pH 12-14 to obtain THCa in aqueous phase; (e) washing the aqueous phase of (d) with the C5-C8 hydrocarbon solvent, and obtaining the aqueous phase; (f) acidifying the aqueous phase obtained in (e) to pH 3-6 to remove metal ion from THCa; and (g) extracting the acidified aqueous phase with the C5-C8 hydrocarbon solvent to obtain a solvent phase rich in THCa from which purified THCa is removed.

The C5-C8 hydrocarbon solvent used in the process may be a C6 or C7 hydrocarbon solvent (hexane or heptane). The hydrocarbon solvent used in each of steps (a), (e), and (g) may be the same or different. Thus, the solvent used may be selected independently in each of steps (a), (e), and (g).

The acid used in step (a) may be 0.1% glacial acetic acid.

The process may additionally comprise washing the organic phase rich in THCa obtained in step (g) with water to remove THCa therefrom. Further, after step (g) the THCa may be purified by chromatography. The chromatography procedure used may comprise silica gel column chromatography with a constant elution mixture of 1.5% acetone in hexane.

During the process, an aqueous phase of the process can be subjected countercurrent centrifugation with an oil. The oil used may comprises a medium chain triglyceride (MCT) oil.

The metal ion in step (c) can be Na+ or K+. For example, the weak metal ion solution in step (c) can be a sodium bicarbonate solution. Such a sodium bicarbonate solution may comprise about 5% sodium bicarbonate.

The acidifying step (f) may comprise addition of a strong acid to reach a pH of about 4-5 in the aqueous phase. For example, acidifying in step (f) may comprise addition of 0.5 M HCl to reach a pH of about 4.5 in the aqueous phase.

The Cannabis plant material may comprise THCa at a level of at least 2% of total cannabinoids. For example, the Cannabis plant material may comprise THCa at a level of at least 5% of total cannabinoids.

The weight ratio of plant material to solvent in the mother liquor of step (a) may be reduced to about 1:3 to about 1:10. Further, the mother liquor of step (a) may be reduced by about 75% of its volume.

In some embodiments, trace amounts of C5-C8 hydrocarbon solvent can be removed from purified THCa after step (g) by formation of a positive binary azeotrope with methanol.

According to certain embodiments described herein, a process for purification of tetrahydrocannabinolic acid (THCa) from Cannabis plant material comprises the steps of: milling Cannabis plant material to form a finely divided Cannabis plant material; extracting the finely divided Cannabis plant material with a C6 or C7 hydrocarbon solvent comprising 0.1% glacial acetic acid to produce a mother extraction liquor; reducing the mother extraction liquor under reduced atmosphere to remove 75% of the solvent, thereby producing a reduced mother liquor; washing the reduced mother liquor with distilled water and separating phases to obtain a washed mother liquor; treating the washed mother liquor with an aqueous 5% sodium bicarbonate solution and separating the phases to obtain a treated mother liquor; extracting the treated mother liquor with a 0.1 M solution of NaOH and separating phases to obtain an aqueous solution comprising THCa sodium carboxylate salt; washing the aqueous solution comprising THCa sodium carboxylate salt three times with the C6 or C7 hydrocarbon solvent and separating to obtain an aqueous solution of washed THCa sodium carboxylate salt; acidify the aqueous solution of washed THCa sodium carboxylate salt to pH 2.5-4, layer with the C6 or C7 hydrocarbon solvent and separating the phases to obtain an organic solution comprising THCa salt; and washing the organic solution comprising THCa salt with distilled water and separating the phases to obtain a solvent phase rich in THCa.

In this embodiment, further isolation of THCa from the solvent phase rich in THCa using chromatography may be conducted so as to obtain THCa of at least 99% purity.

The hydrocarbon solvent can be heptane or hexane in this embodiment, and may be selected independently for the extracting steps.

The process may involve pre-treatment of Cannabis plant material prior to preparation of the mother liquor. The Cannabis plant material may be subjected to an aqueous extraction comprising: (i) soaking the plant material in an acidified aqueous solution and draining; (ii) mixing the plant material of (i) with an aqueous sodium bicarbonate solution and draining; (iii) mixing the plant material of (ii) with a basic solution at pH 12-14, removing biomass therefrom, and collecting the basic aqueous solution; (iv) acidifying the basic aqueous solution from (iii); and (v) preparing the mother liquor by extracting the solution from (iv) in step (a).

An exemplary pre-treatment process for Cannabis plant material involves an aqueous extraction comprising: (i) soaking the plant material in an acidified aqueous solution comprising acetic acid at pH 3.5-5.5, and draining; (ii) mixing the plant material of (i) with an aqueous sodium bicarbonate solution at pH from 7.5-9 and draining; (iii) mixing the plant material of (ii) with a basic solution at pH 12-14 comprising a metal hydroxide or ammonium hydroxide, removing biomass therefrom after contact for 4 to 36 hours, and collecting the basic aqueous solution, optionally purifying said solution by cold filtration or centrifugation; (iv) acidifying the basic aqueous solution from (iii) and removing wax sediment or residual oils therefrom; and (v) preparing the mother liquor by extracting the solution from (iv) in step (a), wherein said solution from (iv) comprises solubilized THCa.

The biomass removed in step (iii) of such a pre-treatment may be composted at or near a location where the Cannabis plant material is grown or obtained.

The draining in steps (i) and (ii) may comprise passage through a dewatering screw.

A process is described herein for preparation of a triglyceride oil comprising tetrahydrocannabinolic acid (THCa) from Cannabis plant material. Preparation of such an oil comprises the steps of: (a) preparing a mother liquor from Cannabis plant material extracted with a C5-C8 hydrocarbon solvent with 0.005% to 0.2% acid by volume; (b) washing the mother liquor with an aqueous solution; (c) treating the washed mother liquor with a weak metal ion solution, (d) extracting the treated mother liquor of (c) with a basic solution of NaOH at pH 12-14 to obtain THCa metal salt; (e) washing the aqueous phase of (d) with the C5-C8 hydrocarbon solvent, and obtaining the washed aqueous phase; and (f) extracting the washed aqueous phase of step (e) with a triglyceride oil to obtain the triglyceride oil comprising THCa.

The washing of the aqueous phase in step (e) may comprise washing with three diminishing measures of fresh non-polar C5-C8 hydrocarbon solvent, pooling these washes, and subsequently removing hydrocarbon solvent under reduced atmosphere.

The extracting of the washed aqueous phase in step (f) may comprise one or more of the following options: extracting the aqueous phase with a medium or long chain triglyceride; shear mixing and settling; conducting countercurrent liquid-liquid centrifugal contacting; and/or liquid membrane extraction. Further, step (f) may additionally comprise filtering the THCa-containing triglyceride phase with activated charcoal, removal of trace organic solvent, and/or further organic solvent extraction.

The process may involve a C6 or C7 hydrocarbon solvent as the indicated C5-C8 hydrocarbon solvent comprises a C6 or C7 hydrocarbon solvent. It may be that the same C5-C8 hydrocarbon solvent is used in steps (a) and (e), or a different solvent can be used in steps (a) and (e). The C5-C8 hydrocarbon solvent may be selected independently in steps (a) and (e).

The acid in step (a) may be 0.01%-0.1% glacial acetic acid.

The process may involve, in step (f), a countercurrent centrifugation with the triglyceride oil. The triglyceride oil comprises a medium chain triglyceride (MCT) oil. Exemplary MCT oils may comprise caproic (C6), caprylic (C8), capric (C10), lauric (C12) fatty acids. For example, a coconut oil or palm kernel oil are exemplary oils comprising MCTs.

The metal ion in step (c) may be Na+ or K+, and an exemplary weak metal ion solution in step (c) may be a sodium bicarbonate solution.

The Cannabis plant material used in the process may comprise THCa at a level of at least 2% of total cannabinoids. The weight ratio of plant material to solvent in the mother liquor of step (a) may be reduced to a level of about 1:3 to about 1:10. For example, the mother liquor of step (a) may be reduced by about 75% of its volume prior to step (b).

The hydrocarbon solvent phase resulting from washing step (e) is discarded or recycled for re-use in step (a) or (e).

An exemplary process for purification of tetrahydrocannabinolic acid (THCa) from Cannabis plant material may comprise the steps of: milling Cannabis plant material to form a finely divided Cannabis plant material; extracting the finely divided Cannabis plant material with a C6 or C7 hydrocarbon solvent comprising 0.1% glacial acetic acid to produce a mother extraction liquor, optionally conducting a pre-processing aqueous extraction of the plant material so as to discard and potentially compost plant biomass prior to preparation of the mother liquor; reducing the mother extraction liquor under reduced atmosphere to remove 75% of the solvent, thereby producing a reduced mother liquor; washing the reduced mother liquor with distilled water and separating phases to obtain a washed mother liquor; treating the washed mother liquor with an aqueous 5% sodium bicarbonate solution and separating the phases to obtain a treated mother liquor; extracting the treated mother liquor with a 0.1 M solution of NaOH to form THCa sodium carboxylate salt, and separating phases to obtain an aqueous solution comprising the THCa sodium carboxylate salt; washing the aqueous solution comprising THCa sodium carboxylate salt three times with the C6 or C7 hydrocarbon solvent and separating to obtain an aqueous solution of washed THCa sodium carboxylate salt; and extracting the aqueous phase of step (e) with a triglyceride oil to obtain the oil comprising THCa.

Such a pre-processing aqueous extraction that may be conducted prior to preparing the mother liquor, may involve the Cannabis plant material being subjected to an aqueous extraction comprising steps (i), (ii), (iii), (iv) and (v) as described above. Such an aqueous extraction may be conducted at a location that permits the biomass removed in step (iii) to be composted at or near a location where the Cannabis plant material is grown or obtained. The draining conducted in steps (i) and (ii) may comprise, for example, passage through a dewatering screw.

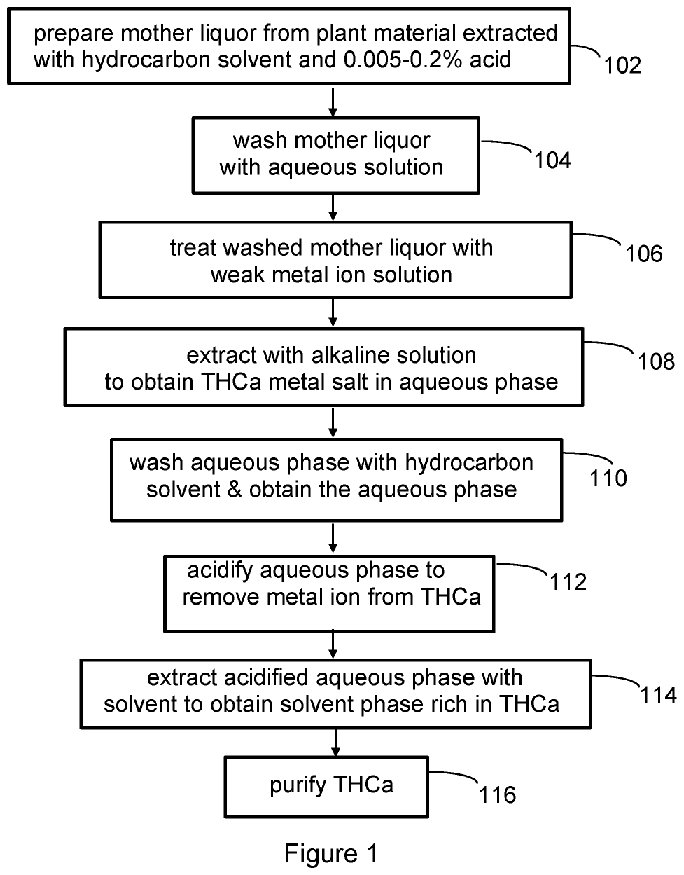

FIG. 1 provides a flowchart depicting the main steps of the process for purification of tetrahydrocannabinolic acid (THCa) from Cannabis plant material involving preparing a mother liquor (102) from finely divided Cannabis plant material in a C5-C8 hydrocarbon solvent, such as hexane, heptane, or pentane, optionally including other hydrocarbon based solvents. The term “mother” liquor is used interchangeably with the term “parent” liquor herein. The hydrocarbon solvent comprises about 0.1% of an acid, such as but not limited to glacial acetic acid, phosphoric acid, maleic acid, tartaric acid, or citric acid.

The mother liquor is washed (104) with an aqueous solution to remove at least a portion of the impurities such as unwanted lipids, fats and waxes by separating non-miscible phases once settled. The washed mother liquor may then be subjected to an aqueous solution of a weak metal ion (106), which pre-treats the mother liquor to enhance the efficiency of subsequent steps, such as by reaction of the carboxylate with sodium bicarbonate or other weaker ionic bases, and separation on non-miscible phases. The step of treating the washed mother liquor with a weak metal ion solution (106) forms a THCa metal salt.

The washed and treated mother liquor is then extracted with a stronger metal ion to move the target carboxylic acid into the aqueous phase and buffer adjusting to pH 12-13 (for example pH 12.65). Extracting with an alkaline solution, permitting the THCa metal salt to be obtained in aqueous phase (108), is conducted with a basic solution of NaOH for example, which effectively reverses the steps of a traditional acid-base extraction. An acid-base extraction is a liquid-liquid extraction. Acid-base extraction is typically used to separate organic compounds from each other based on their acid-base properties. Most organic compounds are more soluble in organic solvents than they are in water. However, some organic compounds can be converted into a water soluble salt. In this case THCa is converted accordingly.

The mother liquor, once rich in THCa, now retains organic impurities, and the THCa carboxylate salt now resides in the aqueous phase. Compound location and intensity may be monitored with thin layer chromatography at this stage.

The spent mother liquor can be re-extracted with additional or other metal ionic solutions such as with potassium hydroxide or ammonia, to form water soluble THCa metal carboxylate salts.

After the aqueous phase is adjusted to a basic pH, for example to a pH of 12.65-13, the result is a basic solution of THCa carboxylate. The aqueous phase is then separated from the spent hydrocarbon solvent phase, and washed (110) with fresh hydrocarbon solvent three times, separating the phase each time to remove trace waxes, and some terpenes. Washing the aqueous phase (110), for example with hexane or heptane, further strips the aqueous phase of unwanted impurities. The hydrocarbon solvent used may be C5-C8, and may be the same or different from the hydrocarbon solvent used in preparation of the mother liquor (102).

Once the aqueous phase has been stripped (110) using C5-C8 solvent, for example hexane or heptane, the obtained THCa rich aqueous phase is layered with fresh hydrocarbon solvent, and is adjusted to an acidic pH (112). Acidifying the aqueous phase to about pH 2-4.5 cleaves the bonded metal ion from the THCa carboxylate salt forming THCa and a side reaction of NaCl and water.

This reaction produces both water and salt. For example, the reaction between solutions of hydrochloric acid and a sodium hydroxide may be represented by the overall equation of: NaOH+HCl→H₂O and NaCl.

Following acidification, the mixture is agitated, the layered hydrocarbon solvent is separated, and the acidified aqueous phase is re-extracted with fresh hydrocarbon solvent (114) (such as C5-C8 solvent, for example hexane or heptane, optionally combined with other non-polar hydrocarbon solvents) to obtain a hexane phase rich in THCa.

Once the solvent phase rich in THCa is obtained, this phase can be further purified, and THCa is removed (116). The solvent phase is then washed once with an aqueous solution to remove polar compounds prior to chromatographic work-up. In instances where multiple solvent phases are obtained, such multiple phases may be pooled prior to or following washing with the aqueous solution.

In an exemplary chromatographic procedure, the organic solvent is stripped, the THCa is picked up in a minimal solvent, loaded onto a normal phase chromatography column packed with silica, and eluted using a strong and weak solvent pairing. Fractions can be identified using UV-VIS detection monitoring 209 nanometers and collected on 228 nanometers. The purified THCa may be, for example of a high +99% purity. The THCa is removed from the solvent by evaporation, treated with MeOH, thus forming a positive binary azeotrope to strip away any residual hydrocarbon impurity, and the liquor is then crystallized to yield powdered form THCa in +99% purity.

Further details of the process are described below.

Carboxylic Acid Salts or Carboxylate Salts

A carboxylic acid salt is the salt formed when a carboxylic acid reacts with a base. Carboxylic acid salts or carboxylate salts are organic compounds that have a —C(═O)O— anionic group. Carboxylate salts have the general formula M(RCOO)ₙ, where M is a metal or a cation like ammonium. The negative charge is delocalized between the oxygen atoms.

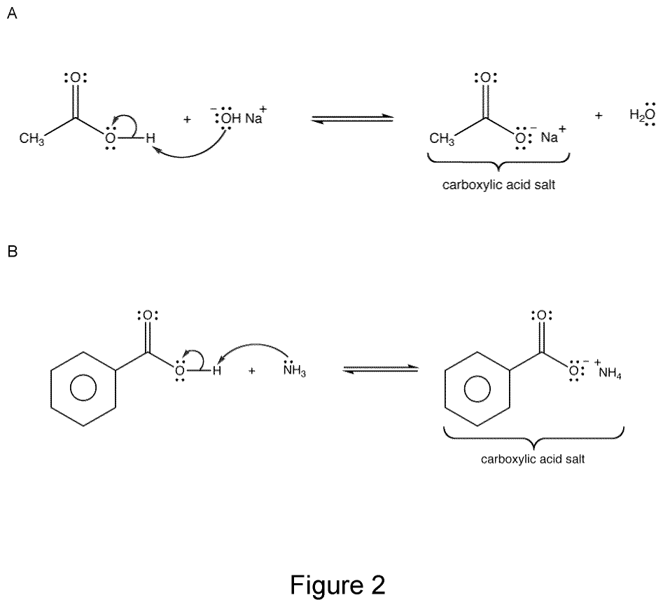

FIG. 2 illustrates two reactions in which carboxylic acid salts or carboxylate salts are formed. Panel A illustrates a sodium carboxylate salt and Panel B illustrates an ammonium carboxylate salt.

Plants Strains and Plant Components. Between different strains of Cannabis and within a single strain, for example within a strain of C. sativa, plants may have different cannabinoid profiles. The process described herein may be used for any strain of Cannabis, and in particular, any strain of C. sativa. A fully matured female plant or a juvenile plant may be used.

A plant strain having a high enough THCa level to warrant extraction is used in the process, such as having THCa as 1% or more of total cannabinoids, preferably at least 2% or at least 5%, for example having a THCa amount of from about 5-10% of total cannabinoids, or higher. With breeding methodologies, higher levels of THCa may be bred in plants, and thus there is no upper limit on the upper amount of THCa as a percentage of total cannabinoids or of major cannabinoids. Other cannabinoid acids, such as CBGa, which may be present in the plant can be separated in the process, and ultimately removed via downstream processing, such as by chromatography. Strains with lower amounts of such cannabinoid acids, other than THCa, may be used.

The dry budding flowers of the plant, or trimmings thereof, are optimally used as the plant material to be extracted. The plant material is dried to an amount of about 10% moisture or less. Prior to processing, harvested plant components may be stored in a sealed, cool, and/or dark environment.

The plant component to be extracted is reduced to smaller sizes such as by chopping, crushing, grinding, or milling upstream of the THCa extraction process described herein. Once the plant components are reduced to a fine consistency to increase surface area in the extraction, and to expose cellular structure, the extraction is conducted.

Exemplary methodologies to prepare plant components to a reduced size for extraction may involve a hammer mill with a 1/16″ or ⅛″ mesh screen. Any methodology for obtaining adequately fine plant material can be utilized.

Optional Pre-Processing Plant Material with Aqueous Extraction Prior to forming Mother Liquor. In certain instances it may be desirable or advantageous to conduct an aqueous extraction of the plant prior to formation of the mother liquor. This may be done at or near the site of plant growth so that large quantities of biomass can be processed instead of shipped to a different processing location. Such an aqueous extraction may involve process the Cannabis plant material by subjecting it to an aqueous extraction.

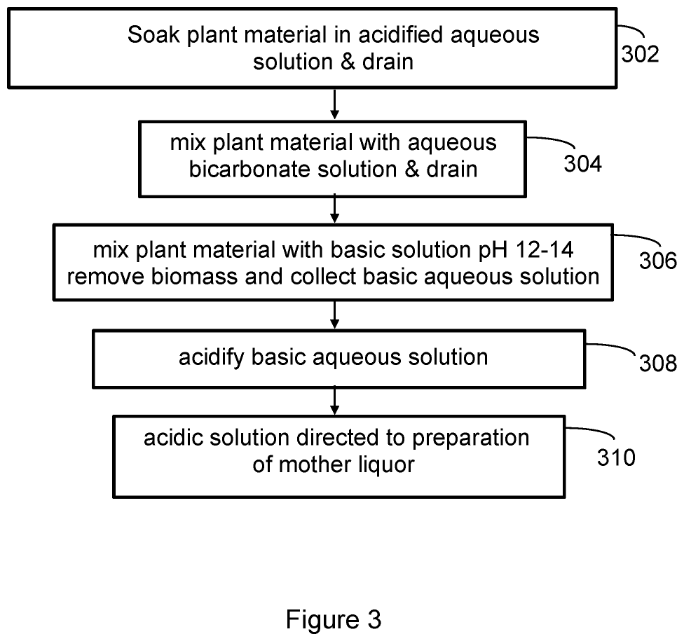

FIG. 3 illustrates such an aqueous extraction which comprises the following steps: soaking the plant material in an acidified aqueous solution comprising acetic acid at pH 3.5-5.5, and draining (302); mixing the soaked and drained plant material with an aqueous sodium bicarbonate solution at pH from 7.5-9 and draining (304); mixing the resulting plant material with a basic solution at pH 12-14 comprising a metal hydroxide or ammonium hydroxide and removing biomass therefrom after contact for 4 to 36 hours, and collecting the resulting basic aqueous solution (306). Optionally a step may be conducted to purify the solution by cold filtration or centrifugation. The resulting basic aqueous solution may be acidified (308), and optionally a majority of the water and/or wax sediment can be removed therefrom. Removed waxes may be redirected for use in other products. The remaining material containing the THCa and other cannabinoid acids can then be directed to preparation of the mother liquor (310) in the downstream steps described herein. Either the plant material or an aqueous extract thereof may be used in the described process.

During the optional pre-processing aqueous extraction steps, the THCa is not water soluble and thus will rise to the top as an oily layer. Most of the water and waxes can be drained away so as to reduce the fluid volume. The remaining material in the reduced volume can thus be extracted with hydrocarbon to thereby produce the mother liquor. Carboxylate salts of THCa, formed later in the process (after the optional pre-processing stage), are water soluble.

In the optional pre-processing steps, the collected wash and treatment solutions contain waxes derived from the plant material, which upon treatment with acid can fall out as sediment and be collected for separate uses, such as in consumer products unrelated to THCa. Acidification of the alkailine solution in the optional pre-processing steps helps to separate the THCa into the oily layer for further processing so that most of the water can be re-used and/or re-directed in an environmentally friendly manner.

The pre-processing may be conducted on a large scale, to reduce biomass on site. Thus, advantageously, an on-site aqueous extraction method can be employed without local use of organic solvent, for example if conducted close to the location of plant growth. Reduction of water from the resulting materials permits a reduced volume to be transported to downstream operations involving a organic solvents, which can then be conducted at a different location.

Pre-processing with an aqueous extraction can advantageously reduce the volume of hydrocarbon solvents required, by reducing the biomass directly acted upon to form the mother liquor. Reduced solvent use has environmental advantages, and reduces energy required in solvent recovery and recycling. The waste water produced from the process can be recycled and reused in the process, with a reduced likelihood of contamination. The acidification of THCa sodium carboxylate, or THCa potassium carboxylate in the optional pre-processing steps allows freed THCa oil to rise to the top. Optionally, centrifugation may be used to assist the separation of a top oily layer this from the water.

Optionally, a large amount of water, such as a majority of the water used in the pre-processing steps, such as at least 50%, 75%, 85%, 90%, 95%, 98% or 99% of the water used in the pre-processing steps can be removed after settling, and the reaming material comprising the small upper aqueous zone and any oils thereon, may be extracted with organic solvent. By only reducing the small upper zone, a lower volume of water is created for recovery and/or recycling after exposure to solvent. A reduced volume of waste water results in a lower likelihood of any environmental contamination as a result of large scale pre-processing.

Extracting Plant Material to form Mother Liquor. The milled plant or the aqueous extraction thereof is subjected to a solvent extract involving a C5-C8 hydrocarbon solvent, such as hexane, heptane, or other suitable solvents, to which a small amount of acid, such as glacial acetic acid (for example about 0.1% by volume) is added or “spiked” so as to render a slightly acidic extraction solvent.

Plant waxes, lignin, and fats can be solubilized in slightly basic solutions or solvents acting as surfactants. Such compounds are considered to be impurities in the process described herein, and can be reduced in the subsequent extraction mother liquor in the presence of a weak acid.

Plant materials are combined in and eluted with this extraction solvent mixture to yield a solution referenced herein as the extraction liquor or “mother liquor” (interchangeably referenced as a “parent liquor”).

The mother liquor is filtered (for example, at 5 micron) during the process to remove small particles of plant materials, to reduce emulsification in subsequent steps or when the phases are mixed. Following filtration, the filtered mother liquor is subjected to further steps of the process. Note that a slightly acidic mother liquor due to addition of a small amount of acid, such as glacial acetic acid, reduces the amount of plant oil and other impurities. Advantageously, oils are left behind, and are thus less prevalent in a slightly acidic C5-C8 (such as hexane or heptane) extraction. The mother liquor is then further processed as described below.

Reduction of Mother Liquor. The mother liquor, filtered and extracted as above, is reduced to a ratio of from 1:3 to 1:10, such as preferably a 1:3 to 1:6 ratio of plant material to solvent (wherein the solvent comprises C5-C8, such; as hexane or heptane, to which 0.1% acid, such as glacial acetic acid, is added). Extraction steps are then conducted on the reduced mother liquor.

For example, a 75% reduction of 100 L of mother liquor would result in a volume of 25 L. In this step, adequate amounts of hydrocarbon solvent are maintained so that the liquor will not be able to hold back undesirable compounds once the pH is adjusted to a basic pH in later steps.

Extraction of THCa from Reduced Mother Liquor via Base:Acid Reaction. The THCa extraction steps utilize a metal ion to form a carboxylate salt of THCa, with the carboxylate salt aspect represented as —RCOO⁻/M⁺ (where “R” is the rest of the THCa cannabinoid molecule). Non-acid cannabinoid moieties do not form the metal salt. This step is conducted as an acid:base reaction, but done in reverse and better represented as a “base:acid” reaction.

The carboxylic acid moiety of THCa is the target monomer for salt formation. Once the salt is formed, water solubility is used to separate the carboxylate salt so formed from other organic substances present in the reduced mother liquor (including other cannabinoids), which remain in the solvent phase that is removed. Upon separation of the water and the hydrocarbon solvent, the THCA⁻/M⁺ salt is separated from the less water soluble cannabinoids, and the carboxylate salts are broken, and the metal removed.

The weaker strength of the metal ion is an advantage in the process as it permits a solubility separation from the non-acid cannabinoids, while later permitting ready removal of the metal ion from the acid moiety of THCa in downstream steps. In this process, the non-acid cannabinoids do not form metal salts, and so remain hydrophobic. Advantageously, the weaker strength of the metal ion, permits all carboxylate salt material to be removed simultaneously.

The cation may be a metal ion, for example sodium (Na+). Sodium is an exemplary metal ion in the base:acid reaction, particularly suitable due to the weaker strength of the acid moiety. Sodium bicarbonate (NaHCO₃) may be used. Sodium is a stable, effective and inexpensive cation, but others (such as other metal salts) may also be suitable, such as those bearing K+ or Mg+ ions. Further, calcium carbonate or an ammonium carboxylate salt, would also be effective.

Stated in terms of an exemplary chemical equations where Na+ is the cation: NaOH+R—COOH═R—COOHNa+H₂O (forming the metal salt), and then R—COOHNa+HCL═R—COOH+NaCl+H (releasing the acid). When the carboxylic acid moieties of THCa form hydrophilic salts, they can enter the aqueous phase of the extraction. The aqueous phase and the hexane phase (containing other cannabinoids and impurities that are not water soluble) are then separated. The aqueous phase is then washed with fresh hydrocarbon solvent and separated prior to acidification.

Acidification. Once THCa is removed into the aqueous phase, the aqueous phase can be further washed or extracted with hydrocarbon solvent to ensure the removal of impurities. For example, a solvent wash with hexane or heptane may be conducted. Once the aqueous phase is of adequate purification, the THCa molecule present in the aqueous phase is acidified and further extraction steps are conducted, as would be used in comparable chemical reactions, to bring about acidification of the mother liquor.

As an example, the addition of a strong acid, such as HCl, to the aqueous phase at a high pH results in a pH of from about 3 to 6, for example from 4 to 5, or preferably around 4.5. A slight molar excess of anion, relative to the amount of cation, may be used. This permits thorough acidification of the THCa salt.

Additional hydrocarbon solvent extractions or washings may be conducted as needed.

Acidification can be conducted at a temperature ranging from 5° C. to room temp, and preferably ambient room temperature conditions permit economical processing costs.

Chromatography and Downstream Purification. Following the extraction and acidification, chromatography with n-hexane may be used as a single solvent system for further purification (specifically: a constant elution mixture of 1.5% acetone in hexane on a silica gel column). Advantageously, hexane is volatile and safe to utilize. Hexane can be removed at low temperatures. Hexane has no structural effect on THCa, and thus cannot convert THCa to THC. Hexane is used as solvent in previous processing steps, and at no previous step does the mixture or extract require evaporation to dryness, as no change in organic solvent is required. Advantageously, normal phase chromatography may be used.

Hydrocarbon solvents, such as hexane, can form a positive azeotrope with methanol so that trace hexane remaining post-processing can be easily removed, before finalizing a purified consumer product. Thus, trace amounts of hexane that may carry through to a methanol azeotrope formation can then be replaced by methanol for crystallization. Thus, it is unnecessary to conduct multiple evaporations of the hydrocarbon solvent.

Other cannabinoid acids (such as CBGa and CBDa) which may be extracted in the process are readily separated out by a chromatographic step and eluted in separate fractions. In such a chromatographic separation, there is no need to include an increasing acetone gradient in the n-hexane form a solvent gradient, which is often a feature of other chromatography systems that use a gradient of solvent strength. Advantageously, chromatography with a constant solvent renders elution bands more compact, resulting in purer fractions. Any other cannabinoid acids isolated in the process may be separated from THCa using chromatography, when present at this stage.

The THCa obtained as a result of the process is highly pure, and as a result of further chromatographic separation can be rendered even more highly purified, at levels about 99% purity.

Other acceptable chromatographic methodologies may be employed according to the process, so as to obtain purified THCa.

Centrifugation and Foam Reduction with Lipid Extraction. Centrifugation can be used to increase processing efficiency of extraction or separation, and to enhance throughput automation.

As cations (such as metals ions) are used to convert THCa to a salt, processing may result in foaming similar to processes used in manufacturing soap from fatty acids. When the sodium carboxylate is formed, it can have a tendency to foam, similar to a soap. This foaming can be mitigated or “calmed” as desired, such as when centrifugation is employed. Undesired foaming at certain stages in the process can be calmed while extracting the subject material according to the process described herein.

A countercurrent centrifuge may be employed, which allows for two non-miscible liquids to run counter current and thereby allowing for washing, treatment, mass transfer, and separation of the liquids therein.

Hydrocarbon solvents used are used at different steps in the extraction, such as in a washing step to remove terpenes from the aqueous solution (prior to acidification and final THCa extraction). In addition to, or as an alternative to the solvent-based washing steps, a lipid (or “oil”) may be employed as a non-miscible solvent with which the aqueous preparation can be further extracted.

In certain embodiments, a triglyceride oil may be used for lipid extraction, wherein the triglyceride oil is not miscible with water (non-polar). The structure of the triglyceride, having three non-polar fatty acids and a glycerol backbone of weak polarity, is suited to the removal of lipid-soluble cannabinoid acids from aqueous solution. Advantageously, introducing a triglyceride phase not only mitigates the foaming of a centrifuged aqueous composition comprising sodium carboxylate, but also serves as an extraction phase following acidification for fat soluble acids to be drawn from the aqueous phase.

Exemplary triglycerides may comprise fatty acids of from 6 to 22 carbons, either unsaturated or saturated to the extent that the triglycerides maintain good fluid movement at processing temperatures. Exemplary fatty acids may be, for example, medium chain triglyceride (MCT) oils which comprise medium chain fatty acid chain lengths from C6-C12. MCT oils may be purified or used in mixtures to achieve desired processing properties, such as fluidity. Triglycerides obtained from plant sources include MCT oils, such as from palm kernel oil or coconut oil.

Optionally, oils so utilized may be back-washed with an organic solvent, such as a hydrocarbon solvent, for additional purity, or can be used to infuse a carrier oil to prepare a less refined but nevertheless market-ready product.

Thin layer chromatography (TLC) confirms the validity of the approach, resulting in high purity fat-soluble fractions that can be readily separated from those of the triglyceride.

Advantageously, such processing permits economical production of a high quality crude oil from the aqueous phase, which crude oil is ready for select markets that do not require a purified isolate. Such processing may be used in addition to or as an alternative to chromatography. Further, countercurrent centrifugation with an oil phase can address undesirable foaming during processing steps.

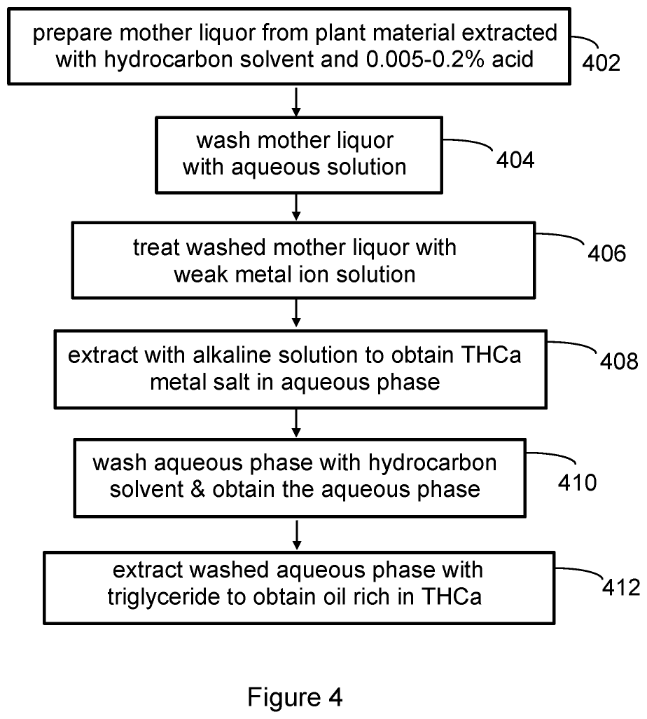

FIG. 4 illustrates a process in which a THCa-containing extract is prepared in a triglyceride oil. The process for preparation of a triglyceride oil comprising tetrahydrocannabinolic acid (THCa) from Cannabis plant material comprising the following steps.

A mother liquor is prepared from Cannabis plant material extracted with a C5-C8 hydrocarbon solvent with 0.005% to 0.2% acid by volume (402). The mother liquor is washed with an aqueous solution (404). The washed and reduced mother liquor is treated with a weak metal ion solution (406). The treated mother liquor is then extracted with a basic solution of NaOH at pH 12-14, to obtain THCa metal salt (408). At this point, mass transfer occurs and the THCa metal salt is in the aqueous phase. The resulting aqueous phase is subsequently washed with the C5-C8 hydrocarbon solvent, and the washed aqueous phase is obtained (410). Subsequently, the washed aqueous phase is extracted with a triglyceride oil to obtain the triglyceride oil comprising THCa (412).

Downstream Processing. THCa obtained by the described method may be processed further, for example, in chemical reactions to form other cannabinoid derivatives in highly purified form. The isolated and/or crystallized THCa can be used as a starting material for other reactions or products, such as in formation of THC by means of a decarboxylation method.

Cannabinoid acids other than THCa can be isolated according to this method. Other cannabinoids can be formed from downstream modifications of the isolated acid product of this method.

### EXAMPLES

The following specific examples outline conditions and possible stepwise methodologies, but should not be construed as limiting.

### Example 1

Extraction of THCa from Cannabis

A mother liquor based on finely chopped Cannabis buds, dried to less than about 10% moisture, is prepared using a hexane solvent comprising 0.1% glacial acetic acid. The mother liquor is subjected to an aqueous extraction in the following steps:

(i) The aqueous phase of the extracted mother liquor is washed with distilled water.

(ii) The washed mother liquor is treated with a weak metal ion sodium bicarbonate aqueous solution (5%) such that sodium serves as the metal cation in the base:acid reaction.

(iii) The aqueous phase thus contains the sodium salt of THCa, and is then extracted with pH 12.6-13.2 of 0.1M NaOH solution (1.1 molar excess).

(iv) The spent mother liquor, so extracted, is then separated in hexane to ensure that the majority to THCa is removed. Spot tests via thin layer chromatography (TLC) are conducted to confirm all target THCa has been captured in the aqueous phase.

(v) The aqueous phase is washed with hexane to remove impurities and ensure separation of impurities from the aqueous phase.

(vi) The washed aqueous phase is then acidified with HCl to achieve a pH of about 4.5. The addition of HCl, a strong acid (0.5M, pH2), is conducted quickly. Good separation of aqueous and organic solvent phases is observed.

The resulting acidified aqueous solution comprising THCa in acid form (Na+ removed) is then extracted with hexane.

(vii) The resulting THCa-rich hexane liquor is then washed with distilled water.

As a result of the base:acid extraction, good separation of THCa from other cannabinoids, oils and other impurities is achieved. Chromatographic separation may then be conducted for further purification.

### Example 2

Extraction of THCa from Cannabis

A mother liquor based on finely chopped Cannabis buds, dried to less than about 10% moisture, is prepared, and THCa is extracted according to the following steps.

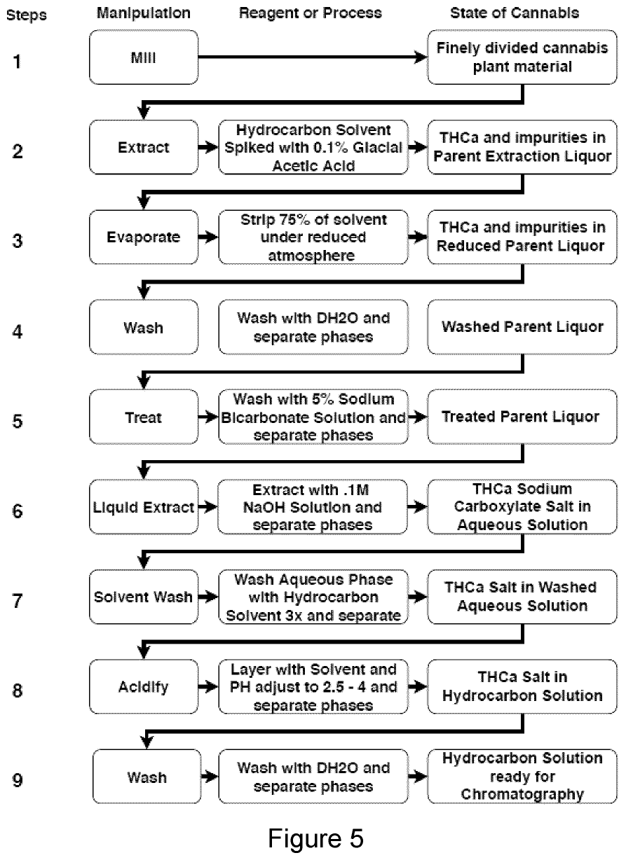

FIG. 5 provides a flowchart to illustrate steps (1) to (9) of the process exemplified herein, for purification of THCa.

(1) Mill chopped Cannabis buds that have been dried to less than about 10% moisture to form finely divided Cannabis plant material.

(2) Extract the milled plant from step (1) with a C6 or C7 hydrocarbon solvent spiked with 0.1% glacial acetic acid, to form a mother liquor.

(3) Reduce the mother liquor to 75% via evaporation at reduced atmosphere to form reduced mother liquor.

(4) Wash the reduced mother liquor with distilled water and separate phases to form a washed mother liquor.

(5) Treat the washed mother liquor with a weak metal ion (sodium bicarbonate 2-5% solution) and separate the phases to form a treated mother liquor. Combine the water wash of step (4) and the aqueous phase separated in order to recapture the mother liquor and combine with the treated mother liquor.

(6) A liquid-liquid extraction is conducted at pH 13 using (0.1 molar) NaOH, KOH, ammonia, or other durable base to form the THCa (metal ion) carboxylate salt. This is done by adjusting the pH in the aqueous solution with 0.25 m buffer pH ≥12.65 and separating the resulting phases.

Step (6) is repeated until TLC indicates that THCa has been adequately removed from the treated mother liquor (from the combined aqueous phases). Optionally, the spent mother liquor, from which all THCa has been depleted, can be further processed to yield terpenes and waxes.

(7) Wash aqueous phase 3 times with a hydrocarbon solvent, which may be the same or different as the C6 or C7 hydrocarbon solvent used in step (2), and separate the washed phase each time. This step removes unwanted components such as waxes, lignin, and terpenes.

(8) Layer aqueous phase with fresh C6 or C7 hydrocarbon solvent and adjust the pH adjust with 0.25-0.5 molar solution of HCl or H₂SO₄ to pH 2.5-4. Mix, settle, and separate. Extract with additional hydrocarbon C6 or C7 hydrocarbon solvent, separate and combine the separated organic phases to obtain a THCa liquor.

(9) Wash the resulting organic phase THCa liquor with distilled water.

Steps (10)-(12) are provided for further purification and isolation of THCa.

(10) Reduce the washed THCa liquor from step (9) to remove solvent under a reduced atmosphere, to thereby produce a reduced THCa liquor.

(11) Load the reduced THCa liquor from step (10) onto a silica column. Run normal phase chromatography monitoring and collecting with UV-Vis detector to yield high purity fractions.

(12) Crystallize primary fractions of THCa.

### Example 3

Aqueous Pre-Processing of Plant Material

In this example, an aqueous (non-solvent) soak is used as an initial pre-processing step. Advantageously, this permits an increase in the scale of biomass being processed at or close to where the biomass is generated. Further, this reduces the amount of hydrocarbon contaminated waste water generated, and the energy required to separate and recover water and solvent tor reuse. Further, the pre-processing allows the process to generate a smaller volume of hydrocarbon mother liquor, which can be processed off site.

Advantageously, this pre-processing step which has the potential to leave much of the unnecessary plant biomass material, such as fibrous materials and lignins at the growing site, thereby saving on transportation costs to locations where a processing plant is located. Removal of biomass near the growing site has the added benefit of allowing low value material to compost in an agricultural setting without requiring transportation.

In this exemplary aqueous extraction, the following steps are conducted:

Coarsely milled or freshly harvested Cannabis is washed using large scale agricultural methods employed to harvest and wash similar plants having comparable biomass.

In a large scale vat, with agitation, biomass is flooded with warm non-iodized distilled water, or reverse osmosis (RO) water spiked with acetic acid to pH 4.5. The mixture is gently agitated and passed through a dewatering screw.

Advantageously, liquid collected from the dewatering screw is an organic and rich “tea” that can be collected and stored for application back into the soil for irrigation and as a fertilizer.

The biomass is then treated by combining with a warm aqueous solution of 5% sodium bicarbonate, and mixed in the vat.

The treated biomass is passed through the dewatering screw, collecting the <pH 8.5 solution discharge, which may be returned to the soil, recycled, or treated as waste water.

The de-watered biomass is then adjusted to about pH 13 solution, using either a metal ion hydroxide, or ammonium hydroxide solution in the vat. The mixture is then agitated and allowed to rest for a number of hours, such as for example 24 hours.

The biomass is then agitated, and biomass is separated from the liquid portion, which contains the desired compound THCa, as well as other cannabinoid acid compounds and other components that were drawn into the liquid.

Optionally, the dewatered biomass may be composted, and thus is useful for application to cultivation fields. Transport of biomass away from an agricultural region to a further processing/industrial region is thus avoided.

The resulting aqueous liquid can be cold filter and/or centrifuged for added purity. The THCa has largely been drawn into this liquid (referenced herein as “mass transfer” having taken place out of the biomass and into the liquid. The biomass may thus be referred to as “denatured”.

Following any desired filtration or centrifugation, the liquid is agitated, and the pH is adjusted to 4.5. The liquid may be allowed to rest prior to further manipulations, so that waxes may precipitate out, while residual oils will rise to the surface, and can be removed. The oil layer may be separated by skimming the liquid surface for example. Further, the vat/tank or container may be drained above the wax sediment line.

The liquid may then be extracted with non polar solvent (such as heptane or hexane), thus reducing the amount of aqueous phase contaminated with hydrocarbon. Advantageously, this reduces the amount of waste water generated in the process. These materials proceed to generate a mother liquor in further steps of the process.

### Example 4

Triglyceride Extraction for Obtaining THCa from Cannabis without Chromatographic Separation

In this Example, a distillate of medium chain triglyceride (MCT) oil from the described process is produced, which can be used in cannabinoid-containing products that do not require a high (99%+) level of purity. An optional centrifugation step is involved.

Triglyceride extraction is conducted after manipulations of plant materials and solvent extraction, and mass transfer of the THCa component, similar to steps (1) to (8) described in Example 2. Following this, the THCa component is now in aqueous phase.

The aqueous phase is separated for further manipulation, while the organic phase is discarded or reserved for recycling for further use as fresh solvent in the process.

The aqueous phase is then washed with three diminishing measures of fresh organic non-polar solvent. The washes are then pooled, and the remaining organic solvent is stripped under reduced atmosphere to yield terpenes.

The aqueous product of the washes is then extracted with a MCT oil (long chain triglyceride may also be used). The extraction is conducted by shear mixing and settling; countercurrent liquid-liquid centrifugal contacting; and/or liquid membrane extraction. Alternatively, other liquid-liquid contacting methods can be used which ensure full liquid phase mixing and separation.

Separation can be aided or sped up by adding non-polar organic solvent to the mixing contacting process.

The phases are separated, and the spent aqueous phase can then be discarded. The triglyceride has the effect of freeing the THCaNa+ from its metal ionic salt form, and thus it becomes soluble in the triglyceride oil.

Triglyceride bonded THCa can then be worked up or further processed or manipulated. For example, for achieving a broad spectrum of compounds other than THCa, such as other cannabinoid acid forms, the organic solvent may simply be stripped away, and the cannabinoids may be decarboxylated as needed.

The THCa triglyceride phase may be treated or filtered with activated charcoal to decolorize and evaporate organic solvent for a narrow spectrum of terpenes and other cannabinoid fatty acids. To further purify, an exemplary method involves diluting 50:50 with organic solvent (such as heptane or hexane), and decolourization of the phase containing THCa triglyceride. This solvent phase can be eluted over silica with a 0 to 4% gradient of strong (acetone) to weak solvent, heptane or hexane to achieve further purification.

Multiple steps of the liquid-liquid extraction steps may be conducted in the same tanks or different tanks, and may be done in batch or continuous flow processing. Agitation steps may be performed in tanks through means of mechanical agitation, or may be a result of turbulence or laminar flow. Agitation and wash steps may be accompanied by resting periods permitting phase separation or settling. For example, steps involving water washes may be followed by a short rest and collection of the water from the wash in the settling tank.

A weak metal base is added to treat the THCa for stabilization and to increase yields of THCa, occurring at that strong metal base extraction step. Through the described process the THCa carboxylate salt is pulled into the aqueous phase, such that the THCa-rich aqueous phase can be collected. The triglyceride extraction option proceeds without need for further chromatographic purification.

The polar nature of THCa (or that of other cannabinoids in acid form) can be utilized, in that a similar electromagnetic effect that joins positive and negative ions such as THCaNa+, effects the sodium carboxylate salt of the fatty acid THCa.

Long, medium and short chain fatty acids are categorized such according to the number of carbons in the aliphatic chain, with medium chain fatty acids ranging from 6 to 12 carbon atoms. A triglyceride of a select fatty acid contains three fatty acids and a glycerol backbone to which the fatty acids are joined by an esterified linkage. This structure, with the aliphatic portions of the three fatty acids stacked up and joined at one end to the glycerol results in the triglyceride having a polarity, but not enough to be water soluble, with medium or long chain fatty acids. The stacked fatty acids in the triglyceride have positive and negative ends. It is this nature that permits the use of these fatty acid groups in the extraction of the THCa from the aqueous phase, without the use of HCl to buffer the solution back to acidic pH. This effectively steals the Na+ ion to form NaCl. In the presence of triglyceride, the weaker metal carboxylate salt bond is overpowered, and joins the THCa.

This example of MCT triglyceride extraction is an alternative to chromatographic purification of THCa. The THCa obtained in an oil form could be used for products that do not require high purity or further chemical manipulation. Triglyceride extraction may be optionally used with a subsequent residual solvent stripping step, and/or one or more optional decolourization steps.

### Example 5

Extraction of THCa from Plant Material with or without Aqueous Pre-Processing

In this example, an overview of parallel exemplary processes is provided in steps A to H, with the first process being direct solvent extraction of plant material, and the second process being pre-processing of plant material with an aqueous biomass extraction.

A. Finely divided plant material is extracted with C6-8 organic solvent, treated with acid or washed with dilute acidic water to form either an initial mother liquor or a washed biomass.

B. The mother liquor is reduced or the washed biomass is treated with sodium bicarbonate, in which case either a reduced mother liquor or a treated biomass is formed.

C. The mother liquor is washed with water, or the biomass is extracted with an alkaline solution, resulting in a washed mother liquor or a THCa rich aqueous phase derived from the biomass.

D. The mother liquor is treated with 5% sodium bicarbonate, or the aqueous phase is acidified to separate out waxes to the bottom and allow a THCa oily layer to rise to the top. In the first case, a treated mother liquor is formed, and in the second case, an isolate oily layer is formed that may be further treated to have the lower portion drained away to thereby reduce the amount of hydrocarbon contaminated water formed.

E. The treated mother liquor is then extracted with an alkaline solution (mass transfer of the THCa into aqueous phase), or the hydrocarbon mother liquor is separated from the aqueous phase. In the first case the aqueous phase is formed, which contains THCa, and in the second case the initial mother liquor is formed, which is directed to later processing as noted herein in step (B) above.

F. The aqueous phase is washed repeatedly (such as 3 times) with organic solvent to wash away the terpines and other impurities prior to acidification. With each wash, the organic phase is separated each time, while the aqueous phase is collected. The washed aqueous phase yields washed aqueous phase THCaNa+.

G. The aqueous phase is layered with fresh organic solvent and acidified to permit mass transfer the THCa to the organic layer. The organic layer is extracted with the aqueous a second time, pooling the organic phases (containing THCa) or extracting the aqueous phase with triglyceride (this may require acid buffering to below PH 12 and organic to assist). In the first case the product is the initial THCa process liquor and in the second case, the result is a THCa triglyceride in organic solvent.

H. Washing is conducted. In the first case, the THCa process liquor is washed with DH₂O and in the second case, the wash is conducted either by filter over AC and strip residual solvent under reduced atmosphere. In the first case a washed process solvent ready for chromatography is formed. Standard normal phase chromatography loading is used. In the first instance, an eluting process using strong and weak solvent pairing over a gradient and in the second instance solvent or ready to test THCa triglyceride infusion is formed.

In the preceding description, for purposes of explanation, numerous details are set forth in order to provide a thorough understanding of the embodiments. However, it will be apparent to one skilled in the art that these specific details are not required.

The described embodiments are intended as examples only. Alterations, modifications and variations can be made to these embodiments by those of skill in the art. The scope of the claims should not be limited by the particular embodiments set forth herein, but should be construed in a manner consistent with the specification as a whole.

### REFERENCES

US2005/0049298 entitled: Production of a delta 9 tetrahydrocannabinol.
US2013/0079531 entitled: Process for the Rapid Extraction of Active Ingredients from Herbal Materials.
US2014/0248379 entitled: Process For Producing An Extract Containing Tetrahydrocannabinol And Cannabidiol From Cannabis Plant Material, And Cannabis Extracts.
US2016/0243177 entitled: Method For Conducing Concentrated Cannabis Oil To Be Stable, Emulsifiable And Flavorless For Use In Hot Beverages and Resulting Powderized Cannabis Oil.
US2016/0228787 entitled: Method and Apparatus for Extracting Plant Oils Using Ethanol Water.
US2016/0326130 entitled: Process To Extract And Purify Delta-9-Tetrahydrocannabinol.
EP 1385595B1 entitled: Processes for preparing a cannabinoid-rich extract from Cannabis plant material.
WO2002/089945 entitled: Processes And Apparatus For Extraction Of Active Substances And Enriched Extracts From Natural Products.
WO2016/200438 entitled: Process for extraction, separation and purification of cannabinoids, flavonoids and terpenes from Cannabis.
WO2016/064987A1 entitled: Cannabis Extracts And Methods Of Preparing And Using Same.
US2011/0256245 entitled: Methods and compositions of Cannabis extracts.
U.S. Pat. No. 8,445,034 entitled: Systems and methods for producing organic Cannabis tincture.
U.S. Pat. No. 9,649,349 entitled: System and method for producing a terpene-enhanced cannabinoid concentrate.
U.S. Pat. No. 9,815,810 entitled: Method of cannabinoid preservation through crystallization and other crystal structures.

1. A process for purification of tetrahydrocannabinolic acid (THCa) from Cannabis plant material comprising the steps of:
(a) preparing a mother liquor from Cannabis plant material extracted with a C5-C8 hydrocarbon solvent with 0.005% to 0.2% acid by volume;
(b) washing the mother liquor with an aqueous solution;
(c) treating the washed mother liquor with a weak metal ion solution to form THCa metal salt;
(d) extracting the treated mother liquor of (c) with a basic solution of NaOH at pH 12-14 to obtain THCa in aqueous phase;
(e) washing the aqueous phase of (d) with the C5-C8 hydrocarbon solvent, and obtaining the aqueous phase;
(f) acidifying the aqueous phase obtained in (e) to pH 3-6 to remove metal ion from THCa; and
(g) extracting the acidified aqueous phase with the C5-C8 hydrocarbon solvent to obtain a solvent phase rich in THCa from which purified THCa is removed.

2. The process of claim 1, wherein:
the C5-C8 hydrocarbon solvent comprises a C6 or C7 hydrocarbon solvent;
the C5-C8 hydrocarbon solvents used in each of steps (a), (e), and (g) are independently selected to be the same or different;
the acid in step (a) is 0.01%-0.1% glacial acetic acid;
the process additionally comprises washing the organic phase rich in THCa obtained in step (g) with water to remove THCa therefrom;
after step (g) the THCa is purified by chromatography;
the metal ion in step (c) is Na+ or K+;
acidifying in step (f) comprises addition of a strong acid to reach a pH of about 4-5 in the aqueous phase;
the Cannabis plant material comprises THCa at a level of at least 2% or at least 5% of total cannabinoids;
the weight ratio of plant material to solvent in the mother liquor of step (a) is reduced to about 1:3 to about 1:10 by volume;
the mother liquor of step (a) is reduced by about 75% of its volume; and/or
trace C5-C8 hydrocarbon solvent is removed from purified THCa after step (g) by formation of a positive binary azeotrope with methanol.

3. (canceled)

4. (canceled)

5. (canceled)

6. (canceled)

7. (canceled)

8. The process of claim 2, wherein said chromatography comprises silica gel column chromatography with a constant elution mixture of 1.5% acetone in hexane.

9. The process of claim 1, wherein an aqueous phase of the process is subject countercurrent centrifugation with an oil.

10. The process of claim 9, wherein the oil comprises a medium chain triglyceride (MCT) oil.

11. (canceled)

12. The process of claim 1, wherein the weak metal ion solution in step (c) is a sodium bicarbonate solution.

13. The process of claim 12, wherein the sodium bicarbonate solution comprises about 5% sodium bicarbonate.

14. (canceled)

15. The process of claim 2, wherein acidifying in step (f) comprises addition of 0.5 M HCl to reach a pH of about 4.5 in the aqueous phase.

16. (canceled)

17. (canceled)

18. (canceled)

19. (canceled)

20. (canceled)

21. A process for purification of tetrahydrocannabinolic acid (THCa) from Cannabis plant material comprising the steps of:
milling Cannabis plant material to form a finely divided Cannabis plant material;
extracting the finely divided Cannabis plant material with a C6 or C7 hydrocarbon solvent comprising 0.1% glacial acetic acid to produce a mother extraction liquor;
reducing the mother extraction liquor under reduced atmosphere to remove 75% of the solvent, thereby producing a reduced mother liquor;
washing the reduced mother liquor with distilled water and separating phases to obtain a washed mother liquor;
treating the washed mother liquor with an aqueous 5% sodium bicarbonate solution and separating the phases to obtain a treated mother liquor;
extracting the treated mother liquor with a 0.1 M solution of NaOH and separating phases to obtain an aqueous solution comprising THCa sodium carboxylate salt;
washing the aqueous solution comprising THCa sodium carboxylate salt three times with the C6 or C7 hydrocarbon solvent and separating to obtain an aqueous solution of washed THCa sodium carboxylate salt;
acidify the aqueous solution of washed THCa sodium carboxylate salt to pH 2.5-4, layer with the C6 or C7 hydrocarbon solvent and separating phases to obtain an organic solution comprising THCa salt; and
washing the organic solution comprising THCa salt with distilled water and separating the phases to obtain a solvent phase rich in THCa;
optionally further comprising isolation of THCa from the solvent phase rich in THCa using chromatography to obtain THCa of at least 99% purity.

22. (canceled)

23. (canceled)

24. (canceled)

25. The process of claim 1, wherein prior to preparing the mother liquor, the Cannabis plant material is subjected to an aqueous extraction comprising:
(i-a) soaking the plant material in an acidified aqueous solution and draining;
(ii-a) mixing the plant material of (i-a) with an aqueous sodium bicarbonate solution and draining;
(iii-a) mixing the plant material of (ii-a) with a basic solution at pH 12-14, removing biomass therefrom, and collecting the basic aqueous solution;
(iv-a) acidifying the basic aqueous solution from (iii-a); and
(v-a) preparing the mother liquor by extracting the solution from (iv-a) in step (a),
or comprising:
(i-b) soaking the plant material in an acidified aqueous solution comprising acetic acid at pH 3.5-5.5, and draining;
(ii-b) mixing the plant material of (i-b) with an aqueous sodium bicarbonate solution at pH from 7.5-9 and draining;
(iii-b) mixing the plant material of (ii-b) with a basic solution at pH 12-14 comprising a metal hydroxide or ammonium hydroxide, removing biomass therefrom after contact for 4 to 36 hours, and collecting the basic aqueous solution, optionally purifying said solution by cold filtration or centrifugation;
(iv-b) acidifying the basic aqueous solution from (iii-b) and removing wax sediment or residual oils therefrom; and
(v-b) preparing the mother liquor by extracting the solution from (iv-b) in step (a), wherein said solution from (iv-b) comprises solubilized THCa.

26. (canceled)

27. The process of claim 25, wherein:
the biomass removed in step (iii-a) or (iii-b) is composted at or near a location where the Cannabis plant material is grown or obtained; and/or
draining in steps (i-a) and (ii-a) or in steps (i-b) and (ii-b) comprises passage through a dewatering screw.

28. (canceled)

29. A process for preparation of a triglyceride oil comprising tetrahydrocannabinolic acid (THCa) from Cannabis plant material comprising the steps of:
(a) preparing a mother liquor from Cannabis plant material extracted with a C5-C8 hydrocarbon solvent with 0.005% to 0.2% acid by volume;
(b) washing the mother liquor with an aqueous solution;
(c) treating the washed mother liquor with a weak metal ion solution to form THCa metal salt;
(d) extracting the treated mother liquor of (c) with a basic solution of NaOH at pH 12-14 to obtain THCa in aqueous phase;
(e) washing the aqueous phase of (d) with the C5-C8 hydrocarbon solvent, and obtaining the washed aqueous phase; and
(f) extracting the washed aqueous phase of step (e) with a triglyceride oil to obtain the triglyceride oil comprising THCa.

30. The process of claim 29, wherein:
washing the aqueous phase in step (e) comprises washing with three diminishing measures of fresh non-polar C5-C8 hydrocarbon solvent, pooling washes, and removing hydrocarbon solvent under reduced atmosphere;
extracting the washed aqueous phase in step (f) comprises: extracting aqueous phase with a medium or long chain triglyceride, shear mixing and settling, conducting countercurrent liquid-liquid centrifugal contacting, and/or liquid membrane extraction;
step (f) additionally comprises filtering THCa-containing triglyceride phase with activated charcoal, removal of trace organic solvent, and/or further organic solvent extraction;
the C5-C8 hydrocarbon solvent comprises a C6 or C7 hydrocarbon solvent;
the C5-C8 hydrocarbon solvents used in steps (a) and (e) are independently selected to be the same or different;
the acid in step (a) is 0.01%-0.1% glacial acetic acid;
step (f) comprises countercurrent centrifugation with the triglyceride oil;
the triglyceride oil comprises a medium chain triglyceride (MCT) oil; and/or
the metal ion in step (c) is Na+ or K+.

31. (canceled)

32. (canceled)

33. (canceled)

34. (canceled)

35. (canceled)

36. (canceled)

37. (canceled)

38. (canceled)

39. (canceled)

40. The process of claim 29, wherein the weak metal ion solution in step (c) is a sodium bicarbonate solution.

41. The process of claim 29, wherein the Cannabis plant material comprises THCa at a level of at least 2% or at least 5% of total cannabinoids.

42. The process of claim 29, wherein the weight ratio of plant material to solvent in the mother liquor of step (a) is reduced to about 1:3 to about 1:10.

43. The process of claim 42, wherein the mother liquor of step (a) is reduced by about 75% of its volume prior to step (b).

44. The process of claim 29, wherein the hydrocarbon solvent phase resulting from washing step (e) is discarded or recycled for re-use in step (a) or (e).

45. (canceled)

46. The process of claim 29, wherein prior to preparing the mother liquor, the Cannabis plant material is subjected to an aqueous extraction comprising:
(i-a) soaking the plant material in an acidified aqueous solution and draining;
(ii-a) mixing the plant material of (i-a) with an aqueous sodium bicarbonate solution and draining;
(iii-a) mixing the plant material of (ii-a) with a basic solution at pH 12-14, removing biomass therefrom, and collecting the basic aqueous solution;
(iv-a) acidifying the basic aqueous solution from (iii-a); and
(v-a) preparing the mother liquor by extracting the solution from (iv-a) in step (a),
or comprising:
(i-b) soaking the plant material in an acidified aqueous solution comprising acetic acid at pH 3.5-5.5, and draining;
(ii-b) mixing the plant material of (i-b) with an aqueous sodium bicarbonate solution at pH from 7.5-9 and draining;
(iii-b) mixing the plant material of (ii-b) with a basic solution at pH 12-14 comprising a metal hydroxide or ammonium hydroxide, removing biomass therefrom after contact for 4 to 36 hours, and collecting the basic aqueous solution, optionally purifying said solution by cold filtration or centrifugation;
(iv-b) acidifying the basic aqueous solution from (iii-b) and removing wax sediment or residual oils therefrom; and
(v-b) preparing the mother liquor by extracting the solution from (iv-b) in step (a), wherein said solution from (iv-b) comprises solubilized THCa.

47. (canceled)

48. The process of claim 46, wherein:
the biomass removed in step (iii-a) or (iii-b) is composted at or near a location where the Cannabis plant material is grown or obtained; and/or
draining in steps (i-a) and (ii-a) or in steps (i-b) and (ii-b) comprises passage through a dewatering screw.

49. (canceled)
